(19) United States
(12) Patent Application Publication
(10) Pub. No.: US 2026/0198883 A1
(43) Pub. Date: Jul. 16, 2026

(54) INFORMATION PROCESSING APPARATUS, RADIATION IMAGING SYSTEM, METHOD OF OPERATING INFORMATION PROCESSING APPARATUS, AND COMPUTER-READABLE STORAGE MEDIUM

(71) Applicant: CANON KABUSHIKI KAISHA, Tokyo (JP)

(72) Inventor: KENTO OSUMI, Tokyo (JP)

(21) Appl. No.: 19/441,090

(22) Filed: Jan. 6, 2026

(30) Foreign Application Priority Data
Jan. 16, 2025 (JP) 2025-006190

(51) Int. Cl. A61B 6/00

(52) U.S. Cl. CPC A61B 6/542

(57) ABSTRACT

An information processing apparatus includes a setting unit that sets a receptor field of a first radiation imaging apparatus having an automatic exposure control function, wherein the setting unit is configured to set a shape of a first receptor field region of the first radiation imaging apparatus based on a shape of a second receptor field region, and wherein the second receptor field region includes a receptor field region of an automatic exposure control apparatus having the automatic exposure control function and being separate from a radiation imaging apparatus.

### BACKGROUND

### Field of the Technology

The present disclosure relates to an information processing apparatus, a radiation imaging system, a method of operating the information processing apparatus, and a computer-readable storage medium.

### DESCRIPTION OF THE RELATED ART

A radiation imaging apparatus having an AEC (Automatic Exposure Control) function is widely used. The AEC is a function for obtaining a desired image with a minimum dose by detecting a part of a radiation transmitted through a subject by a receptor field, converting the detected radiation into an electric signal, and stopping irradiation of the radiation when the integral value of the electric signals reaches a target value. Conventionally, in order to realize the AEC, an ion chamber (ionization chamber) dedicated to the AEC is generally provided separately from a radiation imaging apparatus between the subject and the radiation imaging apparatus to enable automatic exposure.

In the radiation imaging apparatus having the AEC, a receptor field suitable for an imaged site is arranged. For example, in a case of chest frontal imaging, the right side of receptor field and the left side of receptor field are selected in accordance with both lung fields, and in a case of chest lateral imaging, the center of receptor field is selected.

On the other hand, as an imaging apparatus used for medical image diagnosis or nondestructive testing using a radiation, a detecting apparatus using a matrix substrate having a pixel array in which a switch element such as a TFT (thin film transistor) and a conversion element such as a photoelectric conversion element are combined, and a radiation imaging apparatus have been put into practical use. Such a radiation imaging apparatus is used as a digital imaging apparatus for imaging still images such as general imaging and imaging moving images such as fluoroscopic imaging, for example, in the medical image diagnosis.

Recently, multifunctionalization of such a radiation imaging apparatus has been studied. As one of them, incorporation of a function in which an imaging apparatus specifies irradiation information while a radiation source irradiates the radiation has been studied. Using this function, an accumulated radiation dose can be monitored, and when the accumulated radiation dose reaches a target value, the imaging apparatus can control the radiation source and stop the radiation. Therefore, the AEC function can be realized using by only the radiation imaging apparatus.

When the radiation imaging apparatus incorporating the AEC function as described above is portable, the AEC function can be used in various positions, such as a position on a table. For example, it is possible to use the radiation imaging apparatus incorporating the AEC function in combination with a separate AEC apparatus. For example, in a case where the separate AEC apparatus is attached to a standing frame or a table and the radiation imaging apparatus is taken out from the standing frame or the table and used, a usage method that the AEC function of the radiation imaging apparatus is used is thought.

Furthermore, since the radiation imaging apparatus (AEC sensor) incorporating the AEC function can be used in the various positions due to its structure, a receptor field to be used can be selected from a plurality of receptor fields. Japanese Patent Laid-Open No. 2013-52148 discloses a technique for selecting a receptor field of an AEC sensor based on positional information of a receptor field used in a previous AEC sensor. Thus, the position of receptor field to be used can be changed in accordance with an imaged site, and the receptor field to be used can be easily selected from the position information of receptor field used in the past without selecting the receptor field each time the imaged site is changed.

However, the shape of a receptor field region in the AEC apparatus which realizes the AEC function separately from the AEC sensor may be a shape corresponding to the imaged site, such as a shape corresponding to the lung field or the abdomen. In the technique disclosed by Japanese Patent Laid-Open No. 2013-52148, it is possible to change the position of receptor field to be used among receptor fields having predetermined shapes, but the shape of receptor field region cannot be changed in accordance with the shape of imaged site. Therefore, for example, in an examination in which multiple sites are imaged, it is sometimes necessary to use the AEC sensor and the AEC apparatus having different shapes of receptor field region in combination because

the required shape of receptor field region is different according to the imaged site. Therefore, it is difficult to use the AEC sensor in Japanese Patent Laid-Open No. 2013-52148 as a substitute for the AEC apparatus, and it is necessary to use two apparatuses, the AEC sensor and the AEC apparatus, in combination according to the application, and there is a concern that the number of components in the system will be increased and that an extra cost will be incurred.

### SUMMARY

One embodiment of the present disclosure has been made in view of the above concerns, and provides an information processing apparatus capable of setting a receptor field corresponding to various examination conditions for one radiation imaging apparatus having an AEC function.

An information processing apparatus according to an aspect of the present disclosure comprises a setting unit configured to set a receptor field of a first radiation imaging apparatus having an automatic exposure control function, wherein the setting unit is configured to set a shape of a first receptor field region of the first radiation imaging apparatus based on a shape of a second receptor field region, and wherein the second receptor field region includes a receptor field region of an automatic exposure control apparatus having the automatic exposure control function and being separate from a radiation imaging apparatus.

Features of the present disclosure will become apparent from the following description of embodiments with reference to the attached drawings. The following description of embodiments is described by way of example.

### BRIEF DESCRIPTION OF THE DRAWINGS

FIG. 1 is a diagram for illustrating an example of a schematic configuration of a radiation imaging system according to a first embodiment.

FIG. 2 is a diagram for illustrating an example of a radiation imaging apparatus incorporating an AEC function according to the first embodiment.

FIG. 3 is a diagram for illustrating an example of a controlling apparatus and its surrounding configuration according to the first embodiment.

FIG. 4 is a diagram for illustrating an image of imitation of receptor field regions of the AEC function according to the first embodiment.

FIG. 5 is a diagram for illustrating an example of an image of receptor field regions of the AEC function according to the first embodiment.

FIG. 6 is a diagram for illustrating another example of an image of receptor field regions of the AEC function according to the first embodiment.

FIG. 7 is a diagram for illustrating an example of an association between examination information and receptor field information according to the first embodiment.

FIG. 8 is an operational flow of receptor field setting according to the first embodiment.

FIG. 9 is an operational flow from imaging to stopping a radiation according to the first embodiment.

FIG. 10 is a diagram for illustrating an example of an image of display and selection of a receptor field region according to the first embodiment.

FIG. 11 is a diagram for illustrating another example of an image of display and selection of a receptor field region according to the first embodiment.

### DESCRIPTION OF THE EMBODIMENTS

Embodiments for implementing the present disclosure will now be described in detail with reference to the drawings. However, the dimensions, materials, shapes, relative positions of components, and the like described in the following embodiments can be freely set and be changed according to the configuration of an apparatus to which the present disclosure is applied or various conditions. In addition, the same reference numerals are used in the drawings to denote elements that are identical or functionally similar.

In the following, a radiation imaging system using X-rays as an example of a radiation will be described. However, the radiation may be X-rays or other types of radiation. In the following embodiments, the term “radiation” may include, for example, an electromagnetic radiation such as X-rays and γ-rays, and a particle radiation such as α-rays, β-rays, particle rays, proton rays, heavy ion rays, and meson rays.

It should be noted that, in the present disclosure, the term “imitate” as to a shape of receptor field region means generating and setting a shape of receptor field region similar to a shape of receptor field region of the imitated source by referring to the shape of receptor field region of the imitated source. Therefore, the shape of receptor field region generated by imitating the shape of receptor field region of the imitated source may not be coincident with the shape of receptor field region of the imitated source.

### First Embodiment

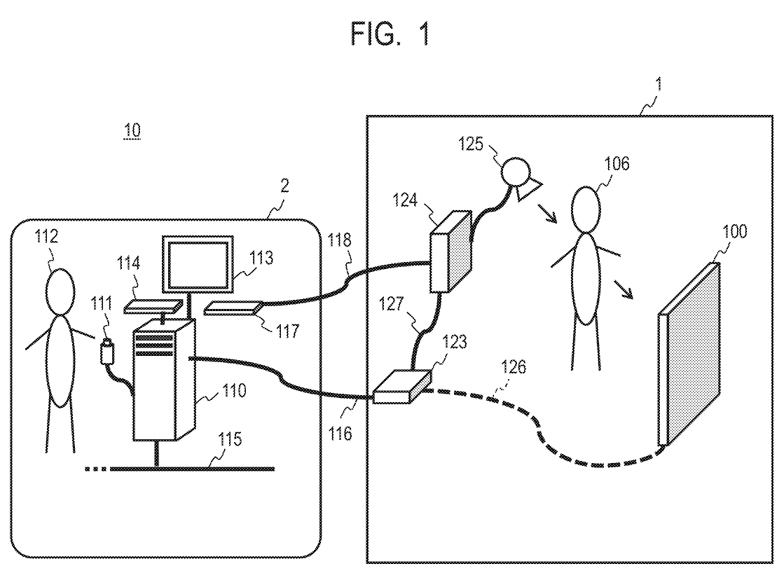

Referring to FIG. 1 to FIG. 11, a radiation imaging system, an information processing apparatus, and a method of operating the information processing apparatus according to a first embodiment of the present disclosure will be described below. FIG. 1 shows an example of a schematic configuration of a radiation imaging system according to the first embodiment.

As shown in FIG. 1, the radiation imaging system 10 is provided in a radiation room 1 for performing radiation imaging by irradiating a radiation and a control room 2 provided near the radiation room 1. The radiation room 1 and the control room 2 are separated from each other in the first embodiment. However, all of the configurations may be provided in the same room as in a case of round imaging.

The radiation room 1 includes a radiation imaging apparatus 100 incorporating an AEC function, a communication controlling apparatus 123, a radiation generating apparatus 124, a radiation source 125, an imaging apparatus cable 126, and a radiation generating apparatus communication cable 127 as components of the radiation imaging system 10. The communication controlling apparatus 123 may wirelessly communicate with the radiation imaging apparatus 100. In the case of wireless communication, the radiation room 1 includes an access point instead of the imaging apparatus cable 126.

The control room 2 includes a controlling apparatus (information processing apparatus) 110, a radiation irradiation switch 111, and a display apparatus 113 as components of the radiation imaging system 10. Further, the control room 2 includes a first input apparatus 114, a second input apparatus 117, an in-hospital LAN 115, a first radiation room communication cable 116, and a second radiation room communication cable 118 as components of the radiation imaging system 10.

The radiation imaging apparatus 100 detects a radiation transmitted through the subject 106 and generates radiation image data. In addition, the radiation imaging apparatus 100 has an AEC function. The configuration of radiation imaging apparatus 100 will be described later.

The communication controlling apparatus 123 controls communication among the radiation generating apparatus 124, the radiation imaging apparatus 100, and the controlling apparatus 110 so that they can communicate with each other.

The radiation generating apparatus 124 controls the radiation source 125 so as to irradiate the radiation based on an irradiation condition corresponding to an examination order or the like. The radiation source 125 includes, for example, a tube, and irradiates the subject 106 with the radiation under the control of the radiation generating apparatus 124.

The imaging apparatus cable 126 is a cable for connecting the radiation imaging apparatus 100 and the communication controlling apparatus 123. The radiation generating apparatus communication cable 127 is a cable for connecting the radiation generating apparatus 124 and the communication controlling apparatus 123.

The controlling apparatus 110 communicates with the radiation generating apparatus 124 and the radiation imaging apparatus 100 via the communication controlling apparatus 123 to integrally control the radiation imaging system 10. The radiation irradiation switch 111, the display apparatus 113, the first input apparatus 114, and the in-hospital LAN 115 are connected to the controlling apparatus 110.

The radiation irradiation switch 111 inputs the timing of radiation irradiation to the controlling apparatus 110 according to the operation of the operator 112. The first input apparatus 114 is an apparatus for inputting instructions from the operator 112, and various input apparatuses such as a keyboard and a touch panel are used. The second input apparatus 117 is an apparatus for inputting instructions from the operator 112 regarding imaging conditions of the radiation generating apparatus 124, and various input apparatuses such as a keyboard and a touch panel are used as in the case of the first input apparatus 114. Only the first input apparatus 114 may be provided. However, an input apparatus capable of exclusively setting imaging conditions of the radiation generating apparatus 124, like the second input apparatus 117, may also be provided.

The display apparatus 113 is an apparatus for displaying image-processed radiation image data and a GUI or the like under the control of the controlling apparatus 110, and any display or the like may be used. The display apparatus 113 may be configured by a touch panel type display. In this case, the display apparatus 113 may also be used as the first input apparatus 114. The controlling apparatus 110, the first input apparatus 114, and the display apparatus 113 may be integrally configured.

The in-hospital LAN 115 is a core network in the hospital. The in-hospital LAN 115 may include an information system and an image management system (not shown).

The first radiation room communication cable 116 is a cable for connecting the communication controlling apparatus 123 in the radiation room 1 and the controlling apparatus 110. The second radiation room communication cable 118 is a cable for connecting the radiation generating apparatus 124 in the radiation room 1 and the second input apparatus 117.

Next, the operation of the radiation imaging system 10 will be described. First, the operator 112 inputs and sets the subject information such as an ID, a name, and date of birth of the subject 106 and the imaging information such as an imaged site of the subject 106 to the controlling apparatus 110 via the first input apparatus 114. The subject information and the imaging information may be set automatically by selecting the examination order received via the in-hospital LAN 115 other than being set by direct input. The subject information and the imaging information can also be set by selecting a preset imaging program. The subject information and the imaging information may be set automatically based on the examination order received from the in-hospital LAN 115 without the operation of the operator 112.

If the imaging preparation is completed, the operator 112 depresses the irradiation switch 111. If the radiation irradiation switch 111 is depressed, after the radiation imaging apparatus 100 performs a desired preparation, the radiation is irradiated from the radiation source 125 toward the subject 106.

The radiation imaging apparatus 100 communicates with the radiation generating apparatus 124 to control the start and end of radiation irradiation. The radiation irradiated to the subject 106 transmits through the subject 106 and enters the radiation imaging apparatus 100. The radiation imaging apparatus 100 converts the incident radiation into visible light and then detects it as a radiation electric signal by a photoelectric conversion element. The radiation imaging apparatus 100 may be configured to directly convert the incident radiation into the radiation electric signal.

The radiation imaging apparatus 100 drives the photoelectric conversion element to read the radiation electric signal and converts an analog signal into a digital signal by an AD converter to obtain digital radiation image data. The obtained digital radiation image data is transferred from the radiation imaging apparatus 100 to the controlling apparatus 110.

The controlling apparatus 110 performs image processing on the received digital radiation image data. The controlling apparatus 110 causes a radiation image based on the image-processed radiation image data to be displayed on the display apparatus 113. The controlling apparatus 110 functions as an image processing apparatus and a display controlling apparatus. The above is the operation of the radiation imaging system 10.

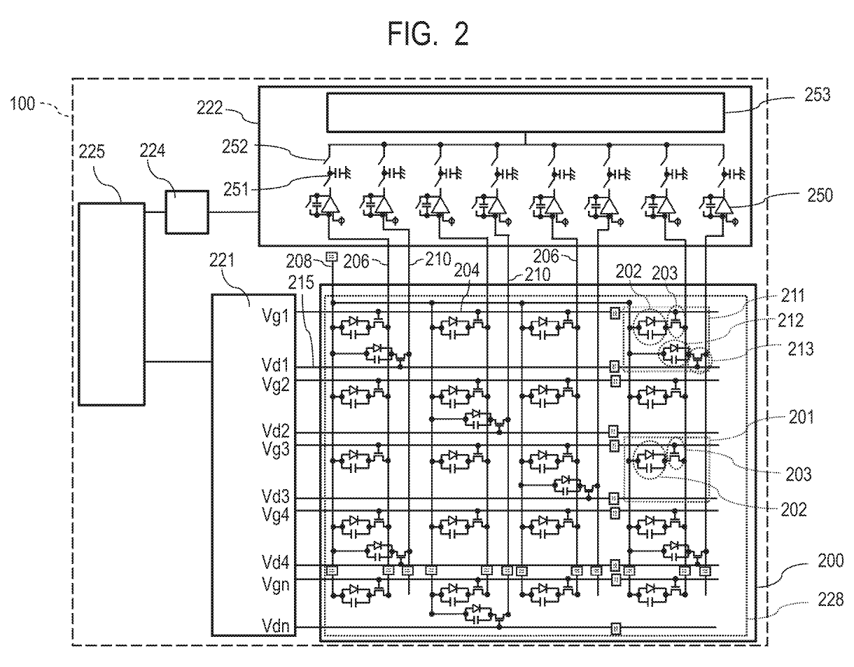

Next, the radiation imaging apparatus 100 incorporating the AEC function will be described with reference to FIG. 2. The radiation imaging apparatus 100 includes a supporting substrate 200 on which a pixel array 228 is arranged, a drive circuit 221, a readout circuit 222, a signal processing unit 224, and an imaging apparatus controlling unit 225. The pixel array 228 includes a plurality of pixels arranged in a matrix. The plurality of pixels includes a first pixel 201 and a second pixel 211.

The first pixel 201 includes, in order to obtain a radiation image, a conversion element 202 for converting the incident radiation or the light into charges corresponding to an incident amount and a switch element 203 for outputting the charges generated by the conversion element 202 to a signal line 206. The conversion element 202 may be an indirect-type conversion element using, for example, a scintillator for converting the radiation into the light and a photoelectric conversion element for converting the light converted by the scintillator into the charges. Further, a direct-type conversion element for directly converting the radiation into the charges may be used as the conversion element 202. A thin film transistor (TFT) using, for example, amorphous silicon or polycrystalline silicon may be used as the switch element 203. For example, polycrystalline silicon may be used according to characteristics required for the TFT. A semiconductor material used for the TFT is not limited to the silicon, but may be other semiconductor materials such as germanium or compound semiconductor.

A first main electrode of the switch element 203 is electrically connected to a first electrode of the conversion element 202, and a bias line 208 is electrically connected to the second electrode of the conversion element 202. The bias line 208 is commonly connected to second electrodes of the plurality of conversion elements 202 arranged along the row. A common bias voltage is supplied to the bias lines 208 arranged in each row. The bias line 208 receives a bias voltage from a power supply circuit (not shown).

The signal line 206 is electrically connected to a second main electrode of the switch element 203. The second main electrodes of the switch elements 203 of the pixels arranged along the row are commonly connected to the signal line 206. The signal line 206 is arranged for each row of pixels. Each signal line 206 is electrically connected to the readout circuit 222. A drive line 204 is electrically connected to the control electrode of the switch element 203. The drive line 204 is commonly connected to control electrodes of the switch elements 203 of a plurality of first pixels 201 arranged along the row, and gate control voltages Vg1 to Vgn are applied from the drive circuit 221 to the drive line 204.

The second pixel 211 includes, in order to obtain the total amount of the incident radiation dose during the irradiation of radiation, a detection element 212 for converting the incident radiation or the light into the electric charge corresponding to the incident amount and a switch element 213 for outputting the electric charge generated by the detection element 212 to a detection line 210. The second pixel 211 may include the above-described conversion element 202 and switch element 203. The detection element 212 may have the same configuration as the conversion element 202, and the switch element 213 may have the same configuration as the switch element 203.

A first electrode of the detection element 212 is electrically connected to a first main electrode of the switch element 213, and a second electrode of the detection element 212 is electrically connected to the bias lines 208 arranged for each column. Second main electrodes of the switch elements 213 arranged along the column are connected to the detection line 210. Each detection line 210 is electrically connected to the readout circuit 222. The drive line 215 arranged for each row is connected to a control electrode of the switch element 213. Gate control voltages Vd1 to Vdn are applied from the drive circuit 221 to each drive line 215.

As shown in FIG. 2, a plurality of second pixels 211 may be arranged in the imaging region, or for example, only one second pixel 211 may be arranged. If a plurality of second pixels 211 are arranged, the incident radiation dose may be detected by only one of the detection elements 212 of the plurality of second pixels 211 or by the plurality of detection elements 212. Further, the second pixel 211 may not be provided, and the drive line 204 may be driven during the irradiation of radiation to obtain the total amount of the incident radiation dose from the first pixel 201.

In the readout circuit 222, the signal line 206 and the detection line 210 are connected to inverting input terminals of respective operational amplifiers 250. The inverting input terminal of the operational amplifier 250 is connected to the output terminal via a feedback capacitor, and a non-inverting input terminal is connected to any fixed potential. The operational amplifier 250 functions as a charge-voltage conversion circuit. The AD converter 253 is connected to a subsequent stage of the operational amplifier 250 via a sample-and-hold circuit 251 and a multiplexer 252.

The readout circuit 222 configures a digital conversion circuit for converting charges transferred from the conversion element 202 and the detection element 212 of the first pixel 201 and the second pixel 211 into the electric signals of the digital signals via the signal line 206 and the detection line 210. The readout circuit 222 may integrate each circuit or may be individually arranged for each circuit.

The signal processing unit 224 processes the signal read out by the readout circuit 222. For example, the signal processing unit 224 processes the digital signal transferred from the conversion element 202 via the signal line 206 and converted by the readout circuit 222 to generate the digital radiation image data. Further, the signal processing unit 224 can process the digital signal transferred from the detection element 212 via the detection line 210 and converted by the readout circuit 222 to calculate the total amount of the incident radiation dose. The signal processing unit 224 may process the digital signal transferred from the conversion element 202 via the signal line 206 and converted by the readout circuit 222 to calculate the total amount of the incident radiation dose.

The imaging apparatus controlling unit 225 controls components in the radiation imaging apparatus 100 such as the drive circuit 221, the readout circuit 222, and the signal processing unit 224. The imaging apparatus controlling unit 225 can transmit, for example, the digital radiation image output from the signal processing unit 224 to the controlling apparatus 110. The imaging apparatus controlling unit 225 can determine irradiation stop timing based on the total amount of the incident radiation dose output from the signal processing unit 224 and transmit the determined irradiation stop timing to the radiation generating apparatus 124.

In the first embodiment, the first pixel 201 is used to obtain a radiation image, and the second pixel 211 is used to obtain a radiation image and the total amount of incident radiation, but the use of the first pixel 201 and the second pixel 211 is not limited thereto. The first pixel 201 and the second pixel 211 may be used to obtain the radiation image and/or the total amount of incident radiation according to a desired configuration. Further, the pixel array 228 may include a plurality of only the first pixel 201 or the second pixel 211. In this case, each pixel may be used separately for obtaining the radiation image, for obtaining the total amount of incident radiation, or for both of these usages according to a desired configuration.

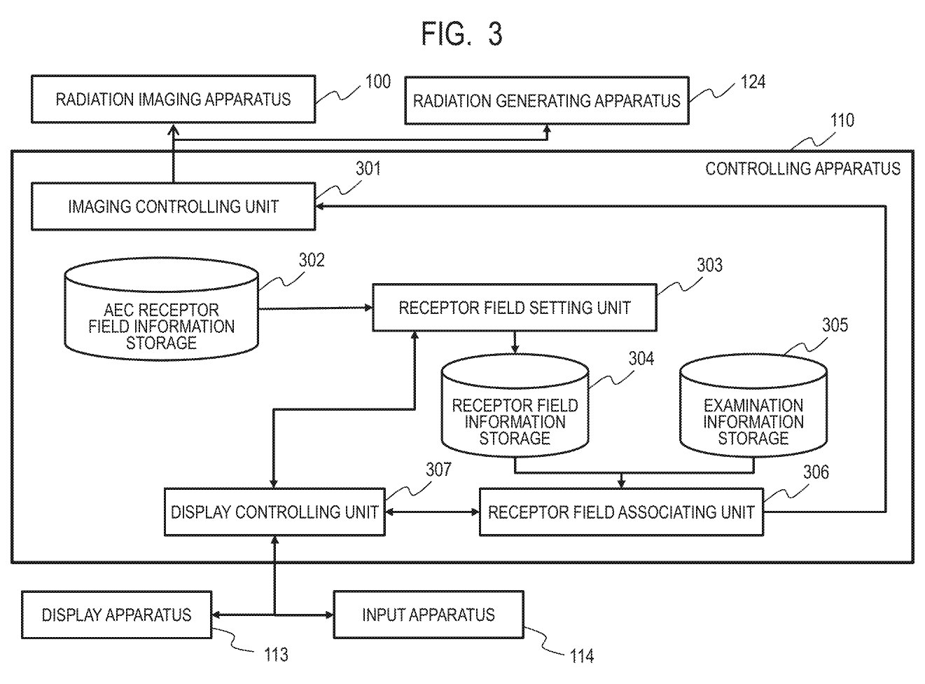

Next, a configuration of the controlling apparatus 110 will be described with reference to FIG. 3. FIG. 3 is a configuration diagram showing an example of a configuration of controlling apparatus 110, and the radiation imaging apparatus 100 and its peripheral apparatuses. The controlling apparatus 110 includes an imaging controlling unit 301, an AEC receptor field information storage 302, a receptor field setting unit 303, a receptor field information storage 304, an examination information storage 305, a receptor field associating unit 306, and a display controlling unit 307.

The imaging controlling unit 301 controls the radiation imaging apparatus 100 and the radiation generating apparatus 124 to control the radiation imaging. The imaging controlling unit 301 exchanges with the radiation generating apparatus 124 information related to the control of radiation generating apparatus 124, for example, notification of the start and stop of radiation irradiation, the AEC information, and the like. The imaging controlling unit 301 also exchanges with the radiation imaging apparatus 100 the radiation image data, the radiation irradiation stop timing, the receptor field information, and the like. The imaging controlling unit 301 can also obtain information on the incident radiation dose from the radiation imaging apparatus 100.

The AEC receptor field information storage 302 stores information for setting a receptor field of the radiation imaging apparatus 100. Here, the receptor field is a region where the incident radiation dose can be detected. The AEC receptor field information storage 302 stores, for example, receptor field information including information on the shape of a receptor field region in an automatic exposure control apparatus (AEC apparatus) separate from the radiation imaging apparatus 100, and receptor field information including information on the shape of a receptor field region included in a radiation imaging apparatus separate from the radiation imaging apparatus 100. The AEC receptor field information storage 302 can also store receptor field information used in the past, information on a region where the subject and the radiation imaging apparatus 100 overlap with each other in the current imaging, and the like.

Further, the AEC receptor field information storage 302 can store information on a combination of the shape of the receptor field region in the AEC apparatus separate from the radiation imaging apparatus 100 and the shape of the receptor field region included in the radiation imaging apparatus separate from the radiation imaging apparatus 100. In this regard, the AEC receptor field information storage 302 can also store information on a shape of a receptor field region in a case where a radiation imaging apparatus different from the radiation imaging apparatus 100 and an AEC apparatus different from the radiation imaging apparatus 100 are used together.

The receptor field setting unit 303 can set a shape of a receptor field region of the radiation imaging apparatus 100. Further, the receptor field setting unit 303 can receive the receptor field information from the AEC receptor field information storage 302 as input, and set the shape of receptor field region of the radiation imaging apparatus 100 by imitating (based on) the shape of receptor field region included in the receptor field information.

As a method of imitating the shape of receptor field region, the receptor field setting unit 303 may, for example, imitate the receptor field region of the receptor field information stored on the AEC receptor field information storage 302 according to an instruction from the operator 112. Further, the receptor field setting unit 303 may perform the imitation based on the position information of receptor field region input from the AEC receptor field information storage 302 without the instruction from the operator 112.

For example, the receptor field information stored on the AEC receptor field information storage 302 is displayed on any display apparatus such as the display apparatus 113, and the operator 112 inputs an instruction to set a receptor field region having a shape similar to the displayed receptor field region. By setting receptor field region according to the instruction from the operator 112, the receptor field setting unit 303 can imitate the receptor field region of the receptor field information stored on the AEC receptor field information storage 302 to set the receptor field region.

Further, AEC receptor field information storage 302 may input, for example, coordinate information of a location where the receptor field region of the stored receptor field information is set to the receptor field setting unit 303 as the input data. In this case, the receptor field setting unit 303 can set the shape of the newly set receptor field region to the same shape as the receptor field region of the input data based on the input data. Note that these methods of imitating the shape of receptor field region are only examples, and the shape of receptor field region may be imitated by other methods.

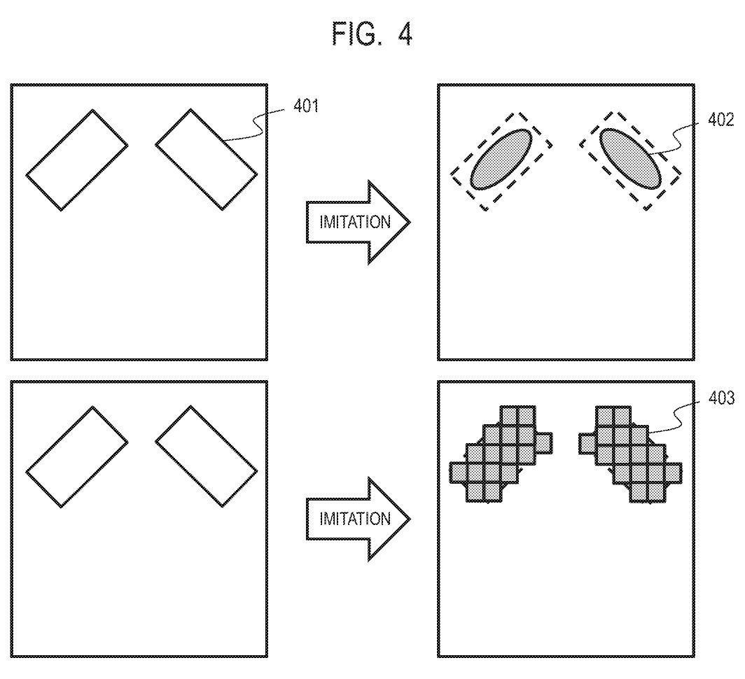

Here, FIG. 4 shows an example of setting the shape of receptor field region in the receptor field setting unit 303 by imitating the information stored on AEC receptor field information storage 302. The receptor field regions 401 in FIG. 4 are the receptor field regions stored on AEC receptor field information storage 302. The receptor field regions 402 show a setting example of receptor field regions which imitate the receptor field regions 401 by the above-described method and are included in receptor field regions 401 in a superimposed manner. On the other hand, the receptor field regions 403 are not included in receptor field region 401, and shows a setting example of imitating the shape of receptor field regions beyond the shape of the receptor field regions 401 as the imitation source.

These setting examples are only examples, and various imitations may be performed in accordance with conditions such as inscription/circumscription, the ratio of overlapping regions, the shape of receptor field region to be set, and the like. Here, as the ratio of overlapping regions, for example, the ratio of overlapping regions between the receptor field region to be set and the receptor field region of the imitation source may be 50% or more. The ratio of overlapping regions is not limited to this, and may be freely set in accordance with a desired configuration.

The receptor field information storage 304 stores the receptor field information of the radiation imaging apparatus 100. Information of receptor field region set by the receptor field setting unit 303 is stored on the receptor field information storage 304 as the receptor field information. The receptor field information stored once on the receptor field information storage 304 can be edited again by the receptor field setting unit 303.

The examination information storage 305 stores examination information related to an examination using the radiation imaging system 10. The examination information stored on the examination information storage 305 may include information related to the imaging such as the patient information, the imaged site information, the imaging method (standing, lying, sitting, portable, etc.), and sensor information to be used (the size and the type of the sensor).

The receptor field associating unit 306 associates the receptor field information stored on the receptor field information storage 304 with the examination information stored on the examination information storage 305. The information to be associated can be freely selected from the examination information stored on the examination information storage 305.

The display controlling unit 307 can control the display apparatus 113 and cause the display apparatus 113 to display, for example, the subject information, the taken radiological image, the examination information, the receptor field region to be set, and the like. The display controlling unit 307 may cause the display apparatus 113 to display the receptor field region of the receptor field information stored on the AEC receptor field information storage 302 and the receptor field region to be set for the radiation imaging apparatus 100 side by side, switched, or superimposed. The display controlling unit 307 may cause the display apparatus 113 to display a GUI for setting the receptor field region according to the instruction from the operator 112 via the first input apparatus 114, and the like.

Here, the controlling apparatus 110 may be configured by a computer provided with a processor and a memory. The controlling apparatus 110 may be configured by a general computer or a computer dedicated to the radiation imaging system. The controlling apparatus 110 may be, for example, a personal computer, a desktop PC, a laptop PC, a tablet PC (portable information terminal), or the like. Further, the controlling apparatus 110 may be configured as a cloud type computer in which some components are arranged in an external apparatus.

The components other than storage of the above-described controlling apparatus 110 may be configured by a software module executed by a processor such as a CPU (Central Processing Unit) or an MPU (Micro Processing Unit). The processor may be, for example, a GPU (Graphical Processing Unit) or an FPGA (Field-Programmable Gate Array). The each component may be configured by a circuit or the like that performs a specific function such as an ASIC. The AEC receptor field information storage 302, the receptor field information storage 304, and the examination information storage 305 may be configured by using any storage medium, for example, any memory, optical storage apparatus, or SSD (Solid State Drive).

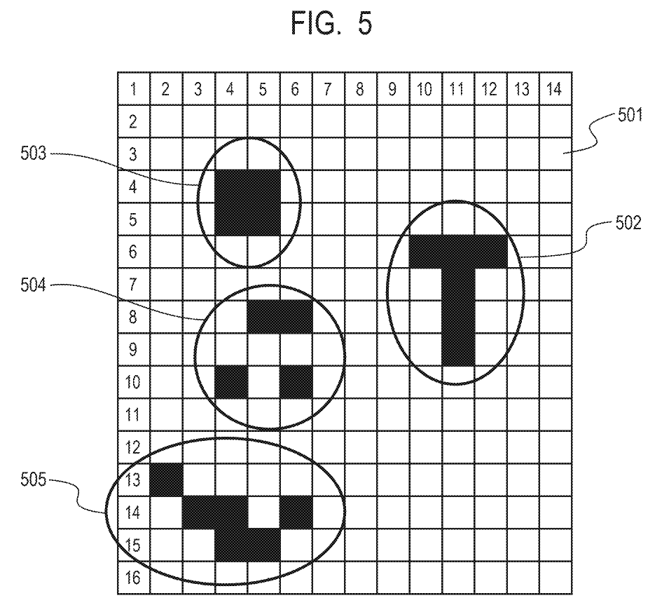

The radiation imaging apparatus 100 can provide a large number of receptor fields in any arrangement and shape, for example, from 5 to the number of pixels of the radiation imaging apparatus 100, with respect to the receptor field, which is usually specified by 1 to 3 points, or 4 points at most. For example, as shown in FIG. 5, it is possible to select a plurality of small receptor field regions 501 in the many receptor field regions of the radiation imaging apparatus 100, and generate large receptor field regions 502~505 by referring to the shape of receptor field regions stored on the AEC receptor field information storage 302. The small receptor field region 501 may correspond to a pixel of the sensor such as the first pixel 201 or the second pixel 211. Note that the receptor fields shown in FIG. 5 is only an example, and a receptor field of any shape, arrangement, and number may be provided. The small receptor field regions to be selected need not be adjacent to each other as shown in the large receptor field region 504 and the large receptor field region 505, but may be obliquely selected and set as the large receptor field region. Note that the large receptor field region may also be referred to as a receptor field group that bundles regions of the minimum unit of receptor field region.

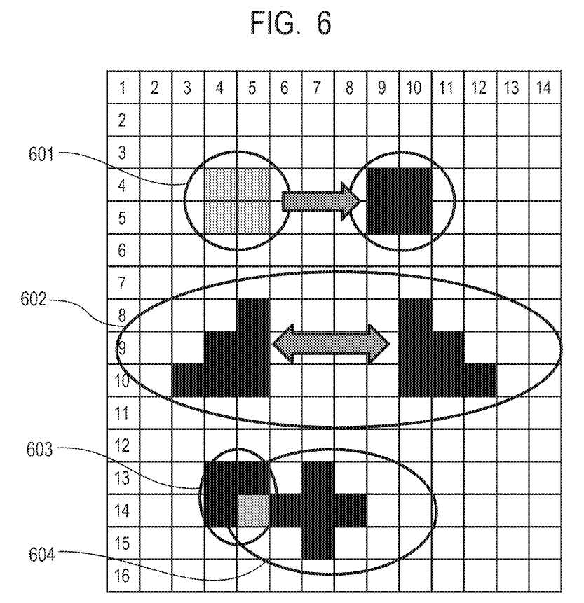

FIG. 6 shows a setting example of another large receptor field region. The receptor field setting unit 303 may set the once set large receptor field region 601 to a position that is translated vertically and horizontally from the once set position by using the coordinate information of pixels arranged in the radiation imaging apparatus 100. Further, like the large receptor field region 602, after generating one large receptor field region, the receptor field setting unit 303 may duplicate and set a large receptor field region that is symmetric with respect to the center of the region in which the receptor field region can be set. In the large receptor field region 603 and the large receptor field region 604, the overlapping region is set as a part of the large receptor field region. In this manner, the receptor field setting unit 303 may also set the overlapping receptor field region as a part of the respective large receptor field regions. Only one small receptor field region is overlapped here, however a plurality of small receptor field regions may be overlapped.

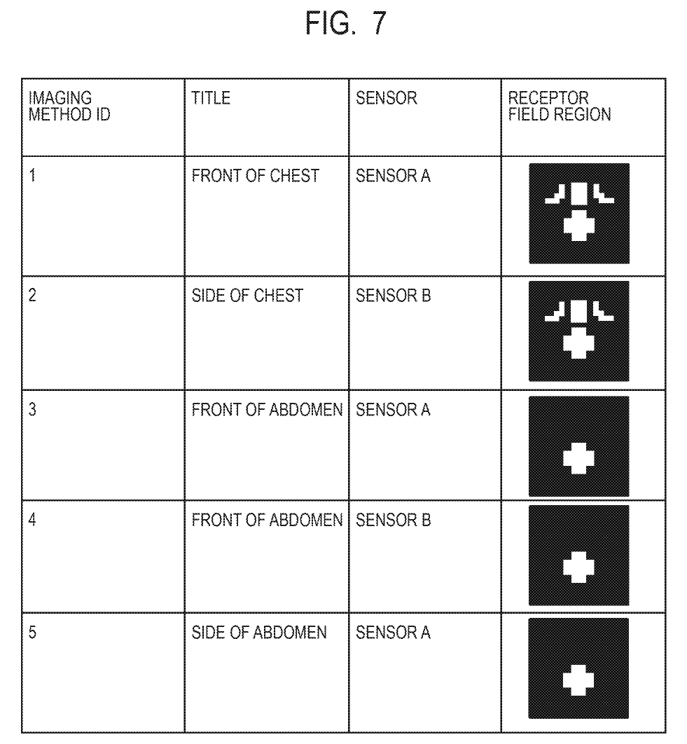

The setting of these receptor field regions is mainly performed before the start of imaging, and the shape of receptor field region prepared in advance is selected and used in accordance with the examination. FIG. 7 shows an example of the receptor field information and the examination information actually associated by the receptor field associating unit 306. For example, in the example shown in FIG. 7, the receptor field information is set in accordance with imaged site. In this example, four receptor field regions are set for the imaging method for the chest being the imaged site, and one receptor field region is set for the imaging method of the abdomen being the imaged site.

In this example, the setting is shown for each imaged site, but the information associated with the receptor field information stored on the receptor field information storage 304 is not limited to this. Other than the imaged site, the receptor field associating unit 306 may associate various information related to the examination, such as the patient information, the radiation imaging apparatus 100 to be used, and the imaging method (standing, lying, sitting, portable, etc.) with the receptor field information stored on the receptor field information storage 304.

The imaging controlling unit 301 is notified of the receptor field information associated by the receptor field associating unit 306, and the imaging controlling unit 301 notifies the radiation imaging apparatus 100 and the radiation generating apparatus 124 of the receptor field information associated with the notified examination information. The radiation imaging apparatus 100 sets the receptor field region on the basis of the notified receptor field information.

The display controlling unit 307 causes the display apparatus 113 to display the receptor field information associated by the receptor field associating unit 306. With respect to the receptor field information displayed on the display apparatus 113, the display controlling unit 307 enables the selection of a receptor field region to be enabled/disabled via the first input apparatus 114. The selected receptor field region may be each small receptor field region or each large receptor field region set in advance.

The display controlling unit 307 causes the display apparatus 113 to display the receptor field information changed by the first input apparatus 114 and notifies the receptor field associating unit 306 of the changed receptor field information. The receptor field associating unit 306 notifies the imaging controlling unit 301 of the receptor field information changed by the first input apparatus 114.

The imaging controlling unit 301 notifies the radiation imaging apparatus 100 of the receptor field information obtained from the receptor field associating unit 306. The imaging apparatus controlling unit 225 of the radiation imaging apparatus 100 controls the drive circuit 221 and the readout circuit 222 based on the notified receptor field information.

The imaging controlling unit 301 controls the radiation generating apparatus 124 based on the information of the incident radiation dose obtained from the imaging apparatus controlling unit 225. The imaging controlling unit 301 and the radiation generating apparatus 124 exchange information related to the control of the radiation generating apparatus 124 such as, notification of the start and stop of radiation irradiation and the AEC information.

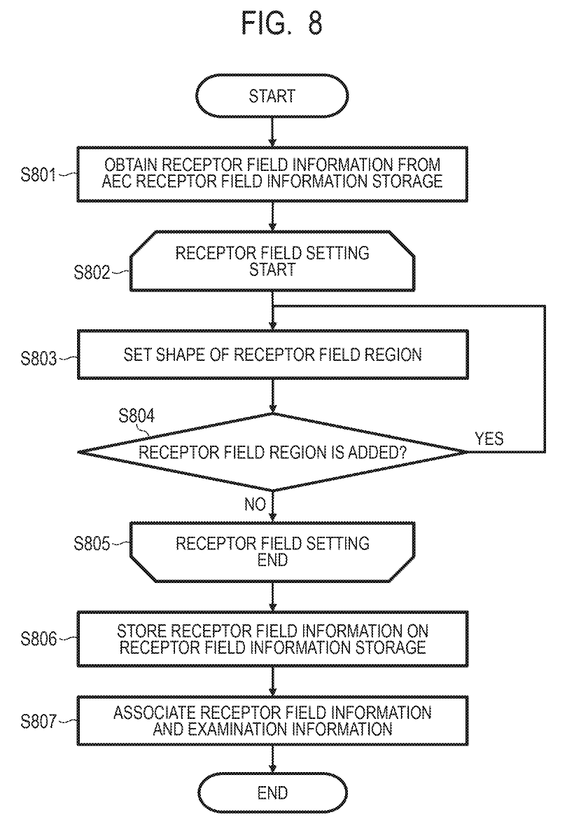

Next, an operation flow of setting the receptor field region using radiation imaging apparatus 100 incorporating the AEC function shown in FIG. 1 with another receptor field information as input will be described with reference to FIG. 8. Hereinafter, an operation flow of setting receptor field region for each imaged site will be described. However, the setting of the receptor field region is not limited to this. The receptor field region may be set for each examination information associated with the receptor field information to be set. The receptor field region may be set, for example, for each imaging method, imaging attitude, imaging direction, presence/absence of a grid, radiation imaging apparatus, or type (sensor type) of radiation imaging apparatus included in the examination information, or may be set for each combined information thereof. The examination information associated with the receptor field region may be other information.

In step S801, the receptor field setting unit 303 obtains receptor field information of another AEC apparatus or radiation imaging apparatus stored on the AEC receptor field information storage 302. At this time, the display controlling unit 307 may cause the display apparatus 113 to display the receptor field information obtained by the receptor field setting unit 303. It should be noted that the receptor field setting unit 303 may obtain from the AEC receptor field information storage 302 the receptor field information corresponding to the examination information associated with receptor field information to be set. In the first embodiment, the receptor field setting unit 303 obtains from the AEC receptor field information storage 302 the receptor field information corresponding to the imaged site.

In step S802, the receptor field setting unit 303 starts the receptor field setting based on the obtained receptor field information of another AEC apparatus or radiation imaging apparatus.

In step S803, the receptor field setting unit 303 sets the shape of receptor field region based on the obtained receptor field information of another AEC apparatus or radiation imaging apparatus. The receptor field setting unit 303 may set the shape of receptor field region in receptor field units. Therefore, the receptor field setting unit 303 may set the shape of one or more receptor field regions for one imaged site, for example.

The shape of receptor field region may be set by the method described above with reference to FIGS. 4 to 6. For example, the receptor field setting unit 303 may set the shape of receptor field region imitating the obtained receptor field information of another

AEC apparatus or radiation imaging apparatus according to the instruction from the operator 112. The receptor field setting unit 303 may also set the shape of receptor field region imitating the obtained receptor field information of another AEC apparatus or radiation imaging apparatus according to a predetermined condition such as the ratio of the inscription/circumscription or the superimposed region. The condition such as the ratio of the inscription/circumscription or the superimposed region may be set for each examination information associated with the receptor field information to be set. However, the setting of the shape of receptor field region is not limited to the above-described method. The shape of receptor field region may be set based on the obtained receptor field information of another AEC apparatus or radiation imaging apparatus by any method in accordance with a desired configuration.

In step S804, the receptor field setting unit 303 determines whether or not to add a receptor field region. This determination may be made in accordance with the instruction from the operator 112, the number of receptor field regions predetermined for each examination information associated with the receptor field information to be set, or the like. If it is determined that the receptor field region is to be additionally set, the process returns to step S803 to set the shape of the next receptor field region. If the setting of all receptor field regions is completed and the receptor field setting is ended, that is, if it is determined that the receptor field region is not to be additionally set, the process proceeds to step S805 to complete the receptor field setting.

In step S806, the receptor field setting unit 303 stores the receptor field information whose setting has been completed on the receptor field information storage 304.

In step S807, the receptor field associating unit 306 associates the receptor field information stored on the receptor field information storage 304 with the examination information stored on the examination information storage 305.

The above-described operation flow is executed as a preliminary preparation before the start of the examination, and the preliminarily prepared receptor field information is used in the examination.

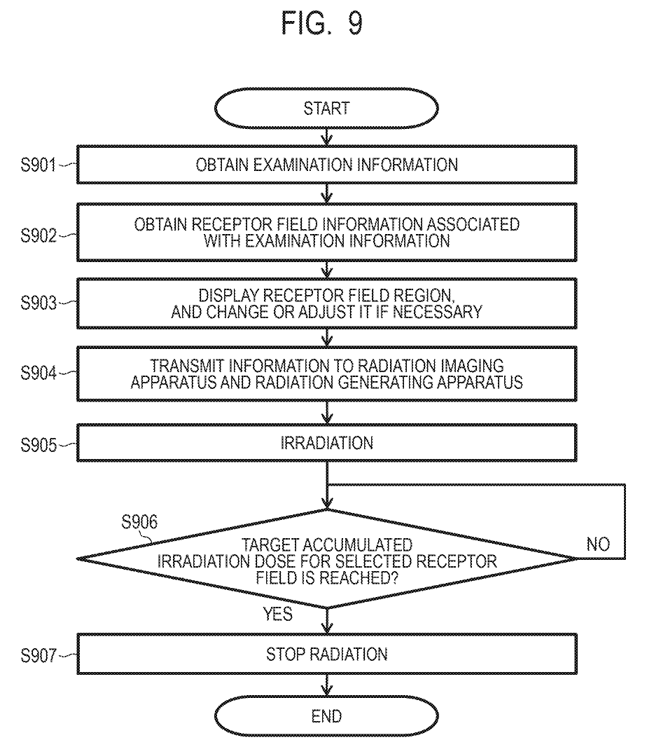

Next, an operation flow when the imaging is actually performed using the receptor field information set in FIG. 8 will be described with reference to FIG. 9. Hereinafter, an operation flow in a case of performing the imaging using the receptor field region corresponding to site information (imaged site) will be described. However, the receptor field region to be used is not limited to the site information, and may correspond to the examination information associated with the preset receptor field information.

In step S901, the controlling apparatus 110 obtains the examination information including the site information, the information on radiation imaging apparatus to be used, and the imaging method such as the standing position, lying position, and free position. The controlling apparatus 110 may obtain the examination information according to an instruction from the operator 112, or may obtain the examination information included in the examination order obtained from the in-hospital LAN 115 or the like.

In step S902, the receptor field associating unit 306 obtains the receptor field information associated with the obtained site information from the receptor field information storage 304. If there is a plurality of pieces of receptor field information associated with the obtained site information, the receptor field associating unit 306 may obtain all of the plurality of pieces of receptor field information.

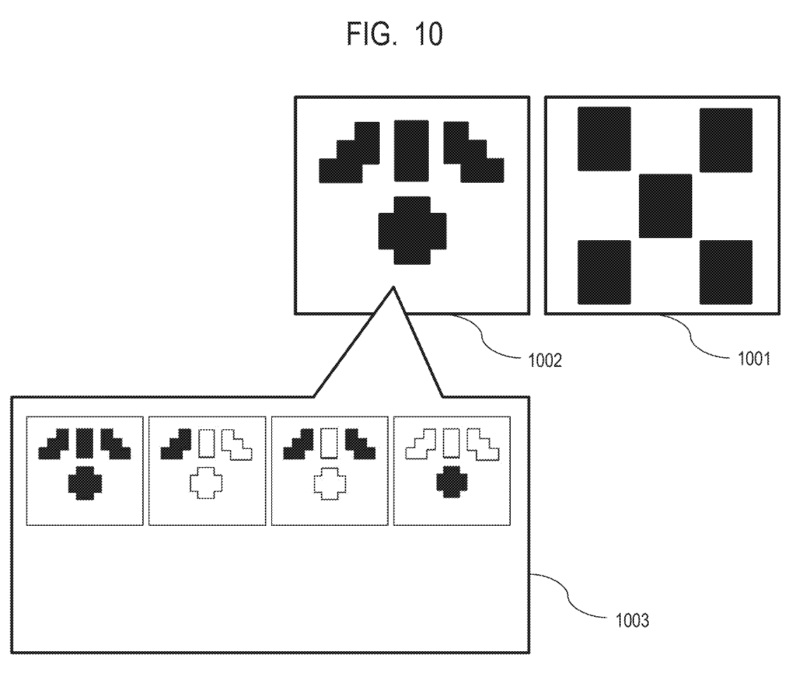

In step S903, the display controlling unit 307 causes the display apparatus 113 to display the receptor field information obtained in step S902. At this time, the display controlling unit 307 may cause to the display apparatus 113 to display the receptor field information in a state where the arrangement of receptor field can be visually recognized. If there is a plurality of pieces of obtained receptor field information, the display controlling unit 307 can cause the display apparatus 113 to display the receptor field information so that the receptor field information can be selected like the receptor field information 1001 and 1002 shown in FIG. 10.

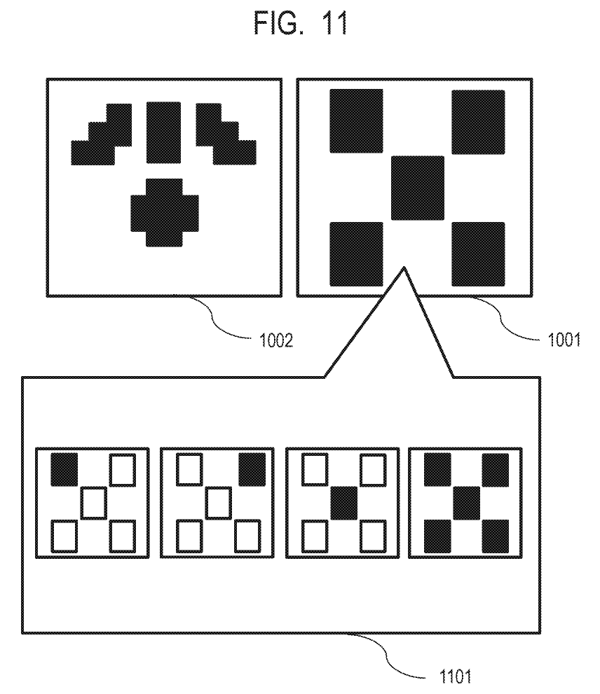

The display controlling unit 307 may also cause the display apparatus 113 to display the receptor field information so that the receptor field region can be changed and adjusted as required. As a method for adjusting the receptor field region, for example, the operator 112 can use the first input apparatus 114 to turn the receptor field region ON or OFF like a toggle by pressing the receptor field of the receptor field information 1001 and 1002 displayed on the display apparatus 113. As a method for changing receptor field region, for example, the display controlling unit 307 may cause the display apparatus 113 to display options of receptor field region set in advance as shown in the window 1003 in FIG. 10 and the window 1101 in FIG. 11, and the operator 112 may use the first input apparatus 114 to select the receptor field region from the options. However, the methods for adjusting and changing the receptor field region are not limited to these, and any method may be used according to a desired configuration.

In step S904, the imaging controlling unit 301 transmits information necessary for performing the imaging, such as information related to the control of the radiation generating apparatus 124 based on the examination information and the AEC information, to the radiation imaging apparatus 100 and the radiation generating apparatus 124. The AEC information transmitted to the radiation imaging apparatus 100 includes, for example, the receptor field information of the AEC, the target accumulated irradiation dose as a threshold value for stopping the radiation, and the like. The information such as the target accumulated irradiation dose may be based on the examination information obtained in step S901.

In step S905, if the imaging preparation is completed and the operator 112 presses the irradiation switch 111, the radiation source 125 irradiates the radiation under the control of the radiation generating apparatus 124. The irradiated radiation transmits through the subject 106 and enters the radiation imaging apparatus 100. The radiation imaging apparatus 100 detects the radiation incident on the receptor field by the pixel selected as the receptor field, and calculates an accumulated irradiation dose that is an accumulated value of the dose (arrival dose) detected in a predetermined period by the signal processing unit 224. The radiation imaging apparatus 100 and the imaging controlling unit 301 are described separately in the first embodiment. However, the imaging controlling unit 301 may be configured in the radiation imaging apparatus 100. The imaging apparatus controlling unit 225 calculates the irradiation dose in the receptor field selected from the accumulated irradiation dose information of each receptor field notified from the signal processing unit 224.

In step S906, the imaging apparatus controlling unit 225 determines whether or not the calculated accumulated irradiation dose of the receptor field reaches the target accumulated irradiation dose. If it is determined that the accumulated irradiation dose reaches the target accumulated irradiation dose, the imaging apparatus controlling unit 225 determines the irradiation stop timing and transmits the determined irradiation stop timing to the imaging controlling unit 301, and the process proceeds to step S907. On the other hand, if it is determined that the accumulated irradiation amount does not reach the target accumulated irradiation dose, the process repeats step S906.

In step S907, the imaging controlling unit 301 notifies the radiation generating apparatus 124 of the irradiation stop of radiation based on the determined irradiation stop timing. The radiation generating apparatus 124 stops the irradiation of radiation by the radiation source 125 based on the notified irradiation stop timing.

As described above, the radiation imaging system 10 according to the first embodiment includes the radiation imaging apparatus 100 for detecting radiation and the controlling apparatus 110 communicatively connected to the radiation imaging apparatus 100. The radiation imaging system may further include the radiation generating apparatus 124 for controlling the radiation source 125 for generating radiation. In this case, the controlling apparatus 110 may be communicatively connected to the radiation generating apparatus 124.

The controlling apparatus 110 may function as an example of an information processing apparatus for controlling the radiation imaging apparatus 100. The controlling apparatus 110 includes the receptor field setting unit 303. The receptor field setting unit 303 can function as an example of setting unit for setting the receptor field of the first radiation imaging apparatus (radiation imaging apparatus 100) having the automatic exposure control function. The receptor field setting unit 303 sets a shape of a first receptor field region of the radiation imaging apparatus 100 based on a shape of a second receptor field region. Here, the shape of the second receptor field region includes a shape of receptor field region of automatic exposure control apparatus (AEC apparatus) having an automatic exposure control function and being separate from a radiation imaging apparatus. The automatic exposure control apparatus may be, for example, an apparatus having an ion chamber.

As described above, the controlling apparatus 110 according to the first embodiment can set the shape of receptor field region of the radiation imaging apparatus 100 based on the shape of receptor field region of the AEC apparatus having a different shape of receptor field region. More specifically, the controlling apparatus 110 can set the shape of receptor field region of the radiation imaging apparatus 100 by matching with (imitating) the shape of receptor field region of the AEC apparatus. As a result, the controlling apparatus 110 can set a receptor field corresponding to examination conditions such as various positions using one radiation imaging apparatus 100. The setting of receptor field region of the radiation imaging apparatus 100 may be performed in advance for the radiation imaging related to the examination.

Further, the shape of the second receptor field region may further include a shape of receptor field region of a second radiation imaging apparatus having an automatic exposure control function and being different from the radiation imaging apparatus 100. Further, the shape of the second receptor field region may further include a combined region of the shape of receptor field region of the automatic exposure control apparatus and the shape of receptor field region of the second radiation imaging apparatus having the automatic exposure control function and being different from the radiation imaging apparatus 100. According to such a configuration, the controlling apparatus 110 can set more variations of receptor fields corresponding to various examination conditions for the radiation imaging apparatus 100.

Further, the receptor field setting unit 303 may set the shape of the first receptor field region by imitating the shape of the second receptor field region. For example, the receptor field setting unit 303 may set the shape of the first receptor field region by imitating the shape of the second receptor field region such that the first receptor field region overlaps the second receptor field region by 50% or more. Further, for example, the receptor field setting unit 303 may set the shape of the first receptor field region by imitating the shape of the second receptor field region such that the first receptor field region circumscribes or inscribes the second receptor field region. According to such a configuration, the controlling apparatus 110 can set the shape of the first receptor field region to be more similar to the shape of the second receptor field region, and can more accurately set a receptor field corresponding to various inspection conditions.

The controlling apparatus 110 can further include an AEC receptor field information storage 302. The AEC receptor field information storage 302 can function as a first storage for storing information of the second receptor field region. The receptor field setting unit 303 may set the shape of the first receptor field region based on the shape of the second receptor field region stored on the AEC receptor field information storage 302. According to such a configuration, the controlling apparatus 110 can set the shape of receptor field region of the radiation imaging apparatus 100 using the information stored on the AEC receptor field information storage 302. As a result, the time required to obtain the shape of the second receptor field region and the time required for the setting process can be shortened.

In the first embodiment, the receptor field setting unit 303 obtains information for setting the receptor field of the radiation imaging apparatus 100 from the AEC receptor field information storage 302. On the other hand, the receptor field setting unit 303 may obtain the information for setting the receptor field of the radiation imaging apparatus 100 from an external apparatus, the in-hospital LAN 115, or the like.

Further, the AEC receptor field information storage 302 may store information of a receptor field region of the automatic exposure control apparatus used in the past as the information of the second receptor field region. According to such a configuration, the controlling apparatus 110 can set the shape of receptor field region of the radiation imaging apparatus 100 by imitating the shape of receptor field region actually used, and can set a more practical shape of receptor field region.

The controlling apparatus 110 may further include the examination information storage 305 and the receptor field associating unit 306. The examination information storage 305 can function as an example of a managing unit that manages examination information. The receptor field associating unit 306 may associate the shape of the first receptor field region set by the receptor field setting unit 303 with the examination information. According to such a configuration, the controlling apparatus 110 may set an appropriate shape of receptor field region based on the examination information. The examination information may include at least one of, for example, the imaged site, the imaging method (standing, lying, sitting, portable, etc.), the imaging attitude, the imaging direction, the presence or absence of a grid, and the sensor type.

The controlling apparatus 110 may further include an imaging controlling unit 301 which can function as an example of a controlling unit for controlling the automatic exposure control function of the radiation imaging apparatus 100 using the information of the first receptor field region associated with the examination information included in an examination order of the subject 106. According to such a configuration, the controlling apparatus 110 can control the automatic exposure control function using an appropriate shape of receptor field region set based on the examination information.

The receptor field setting unit 303 may use one pixel of the radiation imaging apparatus 100 as the minimum unit of receptor field region of the radiation imaging apparatus 100. Further, in a case where a receptor field group is a bundle of regions of the minimum unit of receptor field region of radiation imaging apparatus 100, the receptor field setting unit 303 may set the shape of the first receptor field region using at least one receptor field group. Further, the first receptor field region may include a plurality of receptor field groups which at least partially overlap with each other. Further, the receptor field group may include receptor field regions that are not adjacent to each other. According to such a configuration, the controlling apparatus 110 can more accurately set the receptor field corresponding to various examination conditions.

Further, the receptor field setting unit 303 may set the shape of the first receptor field region by using a first receptor field group and a second receptor field group that is symmetrical with the first receptor field group. Further, the receptor field setting unit 303 may change the coordinate information of the first receptor field region in units of the receptor field group. According to such a configuration, the controlling apparatus 110 can more efficiently set the receptor field region.

Further, the receptor field setting unit 303 may switch between enabling and disabling the receptor field group in the first receptor field region in units of the receptor field group in response to an instruction from the operator 112. According to this configuration, controlling apparatus 110 can more accurately set the receptor field corresponding to various examination conditions. Furthermore, the controlling apparatus 110 can further include the display controlling unit 307 that functions as an example of a display controlling unit that causes the display apparatus 113 to display the information of the first receptor field region. According to this configuration, the operator 112 can more easily understand the information of receptor field region to be set.

According to one embodiment of the present disclosure, a receptor field corresponding to various examination conditions can be set for one radiation imaging apparatus having an AEC function.

### Other Embodiments

Embodiment(s) of the present disclosure can also be realized by a computer of a system or apparatus that reads out and executes computer executable instructions (e.g., one or more programs) recorded on a storage medium (which may also be referred to more fully as a 'non-transitory computer-readable storage medium') to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)^{TM}), a flash memory device, a memory card, and the like.

While the present disclosure has been described with reference to embodiments, it is to be understood that the present disclosure is not limited to the disclosed embodiments. The scope of the following claims is to be accorded the broadest interpretation so as to encompass all such modifications and equivalent structures and functions.

This application claims the benefit of Japanese Patent Application No. 2025-006190, filed January 16, 2025, which is hereby incorporated by reference herein in its entirety.

What is claimed is:

1. An information processing apparatus comprising:
a setting unit configured to set a receptor field of a first radiation imaging apparatus having an automatic exposure control function,
wherein:
the setting unit is configured to set a shape of a first receptor field region of the first radiation imaging apparatus based on a shape of a second receptor field region; and
the second receptor field region includes a receptor field region of an automatic exposure control apparatus having the automatic exposure control function and being separate from a radiation imaging apparatus.

2. The information processing apparatus according to claim 1, wherein the second receptor field region further includes a receptor field region of a second radiation imaging apparatus having the automatic exposure control function and being different from the first radiation imaging apparatus.

3. The information processing apparatus according to claim 1, wherein the second receptor field region further includes a combined region of the receptor field region of the automatic exposure control apparatus and a receptor field region of a second radiation imaging apparatus having the automatic exposure control function and being different from the first radiation imaging apparatus.

4. The information processing apparatus according to claim 1, wherein the setting unit is configured to set the shape of the first receptor field region by imitating the shape of the second receptor field region.

5. The information processing apparatus according to claim 4, wherein the setting unit is configured to set the shape of the first receptor field region by imitating the shape of the second receptor field region such that the first receptor field region overlaps the second receptor field region by 50% or more, or the first receptor field region circumscribes or inscribes the second receptor field region.

6. The information processing apparatus according to claim 1, further comprising:
a first storage configured to store information of the second receptor field region,
wherein the setting unit is configured to set the shape of the first receptor field region based on the shape of the second receptor field region stored on the first storage.

7. The information processing apparatus according to claim 6, wherein the first storage stores information of a receptor field region of the automatic exposure control apparatus used in the past as the information of the second receptor field region.

8. The information processing apparatus according to claim 1, further comprising:
a managing unit configured to manage examination information; and
an associating unit configured to associate the shape of the first receptor field region set by the setting unit with the examination information.

9. The information processing apparatus according to claim 8, wherein the examination information includes at least one of an imaged site, an imaging method, an imaging attitude, an imaging direction, presence or absence of a grid, and a sensor type.

10. The information processing apparatus of claim 8, further comprising:
a control unit configured to control the automatic exposure control function of the first radiation imaging apparatus using information of the first receptor field region associated with examination information included in an examination order of a subject.

11. The information processing apparatus according to claim 1, wherein the setting unit is configured to use one pixel of the first radiation imaging apparatus as a minimum unit of the receptor field region of the first radiation imaging apparatus.

12. The information processing apparatus according to claim 1, wherein, in a case where a receptor field group is a bundle of regions of a minimum unit of the receptor field region of the first radiation imaging apparatus, the setting unit is configured to set the shape of the first receptor field region using at least one of a receptor field group.

13. The information processing apparatus according to claim 12, wherein the first receptor field region includes a plurality of receptor field groups which at least partially overlap with each other.

14. The information processing apparatus of claim 12, wherein the receptor field group includes receptor field regions that are not adjacent to each other.

15. The information processing apparatus according to claim 12, wherein the setting unit is configured to set the shape of the first receptor field region using a first receptor field group and a second receptor field group that is symmetrical with the first receptor field group.

16. The information processing apparatus according to claim 12, wherein the setting unit is configured to change coordinate information of the first receptor field region in units of the receptor field group.

17. The information processing apparatus according to claim 1, further comprising a display controlling unit configured to cause a display apparatus to display information of the first receptor field region.

18. A radiation imaging system comprising:
a radiation imaging apparatus arranged to detect a radiation; and
the information processing apparatus according to claim 1 communicatively connected to the radiation imaging apparatus.

19. A method of operating an information processing apparatus, the method comprising:
setting a receptor field of a first radiation imaging apparatus having an automatic exposure control function, wherein:
the setting includes setting a shape of a first receptor field region of the first radiation imaging apparatus based on a shape of a second receptor field region; and
the second receptor field region includes a receptor field region of an automatic exposure control apparatus having the automatic exposure control function and being separate from a radiation imaging apparatus.

20. A non-transitional computer-readable storage medium having stored thereon a program that, when executed by a computer, cause the computer to execute the method of operating the information processing apparatus according to claim 19.
